(19) United States
(12) Patent Application Publication
(10) Pub. No.: US 2026/0233215 A1
(43) Pub. Date: Aug. 13, 2026

(54) Cultureware with Pipette Resting Ledges

(71) Applicant: XDemics Corporation, Novato, CA (US)

(72) Inventors: Colin A. Cook, Oakland, CA (US); Austin R. Santiago, Novato, CA (US); Nicholas E. Scianmarello, San Francisco, CA (US); Gabriel E. Zwillinger, San Francisco, CA (US); Christopher D. Thanos, Tiburon, CA (US)

(73) Assignee: XDemics Corporation, Novato, CA (US)

(21) Appl. No.: 19/536,252

(22) Filed: Feb. 11, 2026

Related U.S. Application Data
(60) Provisional application No. 63/757,456, filed on Feb. 12, 2025.
(60) Provisional application No. 63/770,927, filed on Mar. 12, 2025.
(60) Provisional application No. 63/874,207, filed on Sep. 2, 2025.

(51) Int. Cl. B01L 3/02; A61B 17/08; B01D 53/22

(52) U.S. Cl. CPC B01L 3/0279; A61B 17/08; B01D 53/22; B01L 3/021; B01L 3/0217; B01L 3/0275; B01L 3/0293

(57) ABSTRACT

Gas permeable cultureware with ledges on their liquid holding area walls are described. Each ledge can have a resting surface for the tip of a pipette as well as a cradle-like area that captures the end of the pipette. A cultureware vessel can have multiple ledges spanning around its inside or placed vertically above and below each other. The ledges can facilitate the addition and removal of liquid media, cells, and waste. The ledges provide a robust area for the operator to rest a pipette tip during operations without risk of puncturing a gas permeable membrane of the device. They can have a spillway that redirects the flow of liquid media to minimize disruption of cells in the cultureware, a visual indicator for the amount of media added to a device, and a weirs for the removal of media to precise heights or volumes within the device.

### CROSS-REFERENCES TO RELATED APPLICATIONS

This application claims the benefit of U.S. Provisional Patent Application No. 63/757,456, filed 12 Feb. 2025, U.S. Provisional Patent Application No. 63/770,927, filed 12 Mar. 2025, and U.S. Provisional Patent Application No. 63/874,207, filed 2 Sep. 2025, the contents of which are hereby incorporated by reference in their entireties for all purposes.

### STATEMENT AS TO RIGHTS TO INVENTIONS MADE UNDER FEDERALLY SPONSORED RESEARCH AND DEVELOPMENT

Not applicable.

### BACKGROUND

### 1. Field of the Invention

The present application generally relates to containers or dishes for laboratory use in retaining material to be analyzed with fluid transport features. Specifically, the application is related to petri dishes, single-and multi-well plates, and other cultureware with physical pockets, hollows, shelves, or other ledge features that allow a user to rest the end of a pipette and assure proper volume or height of liquid media or fluid flow ingress or egress direction with respect to cell-growing geometry on the bottom of the cultureware.

### 2. Description of the Related Art

Petri dishes, single-and multi-well plates, flasks, and other cultureware have been used for over a century by scientists worldwide to cultivate microorganisms, such as bacteria, yeasts, and molds. Cultureware is commonly made of glass or hard plastic and comes in a variety of forms. The particular form factor of cultureware used in experiments depends on the need, size, and its availability. For cell cultivation, a liquid medium is prepared and poured or pipetted into the cultureware. Sometimes the liquid is formulated to harden. Cells are seeded into or onto the medium with a pipette and left to grow.

Pipetting fluid and/or cells into or out of cultureware can be done by hand or by robotic methods, individually or in multichannels. Traditionally, it does not particularly matter exactly where the tip of a pipette is in the cultureware when filling. Each scientist may have his or her own preferences, or an experiment may call for the pipette to be in a standard location. For example, the pipette tip may rest on the bottom, in a corner, or even be freely held above the cultureware basin. Aspirating, by applying a vacuum to a pipette, demands better placement because it must remain underneath the liquid surface, but it too is subject to preference. Often, aspiration is performed at a low point in the cultureware, sometimes aided by a user tilting the entire cultureware vessel.

Cultureware use can be manually burdensome, but it excels for small tests to culture cells that grow best on surfaces rather than in solution.

Stirred tank reactors (STRs) and cell factories have been historically adopted to scale up production of bioproduct for gene therapy. However, as has been repeatedly acknowledged in the literature, demands for Adeno-Associated Virus (AAV) for systemic therapy vastly exceed the production capability of stirred tank reactors.

With increased understanding of the genetic basis of many diseases, the possibility of treatment by gene transfer and gene editing is becoming a clinical reality. Many of these new therapies will depend on viral vectors. Developed cell-based vector production relies on traditional tissue culture technologies. The high dose of viral particles needed for these applications, combined with the inefficiency of current production methods, results in a staggeringly high cost for these medicines.

Cost and production times are among the largest barriers to clinical investigation of promising approaches at the Phase I and II stages. The high cost is because the quantity of viral vector needed for trials necessitates cell culture at the trillion-cell scale (the 10¹² cell scale). With developed technology, production of sufficient vector for early-stage trials may take 9-12 months, and existing manufacturing facilities have been reportedly near maximum capacity. The wait time to have vectors produced and into trials can be 2-3 years, delaying revolutionary therapies from reaching testing and clinical application.

The high cost and long queues in production are delaying translation of potentially disruptive therapies that may alleviate suffering and preserve life. Some promising approaches may end up being abandoned altogether because of overwhelming financial obstacles.

There is a need in the art for improvements in cell and tissue growth production apparatuses and methods that reduce the cost and time of production for viral vectors and facilitating testing and clinical use of promising therapies.

### BRIEF SUMMARY

Generally, laboratory cultureware is described that includes one or more small ledges within its liquid holding area. The ledges are set into or against its internal sidewall. They can have a flat-sided or rounded concave border area, called a “surround,” immediately around it that figuratively, and in some circumstances literally, cradles the end of a pipette. For example, a pipette can rest on the ledge while liquid is input or extracted. If the pipette is bumped, its tip can slide and wedge itself against a corner.

Multiple ledges can be set against a wall at different heights, like an inverted Olympic podium. There may be cradle areas against the wall or no cradle area.

Each ledge can be made of a hard material (e.g., hard plastics such as polystyrene, polycarbonate) or soft materials (e.g., silicone, sometimes molded out of the same silicone as the well and base) while the bottom of the cultureware well can be comprised of a thin, easily puncturable material, such as a gas-permeable membrane. A spillway underneath the ledge can be engineered with facets and features that partially arrest and/or disburse liquid into the basin below. Ledges can be aligned with or against fins or other elongated features in the bottom cell-growing area so as to better shelter or flush cells growing within them.

The ledge can be at a height that indicates to a user a fill height or volume of liquid in the bottom. Different ledges in the same cultureware can have different heights for different fill heights/volumes.

Techniques to use the ledge for filling a consistent amount of liquid media are also described. The cultureware vessel can be filled up with liquid and then a pipette—with its tip resting on the ledge—can aspirate fluid away until the liquid level descends to the pipette's end and suction is broken.

Some embodiments of the present invention are related to a cultureware vessel apparatus including a basin bed, a sidewall surrounding and sealed against the basin bed sufficient to hold liquid, a ledge set against or into the sidewall, the ledge having a horizontal surface above the basin bed, and a concave surround extending around a portion of the ledge, the ledge and the surround forming a three-dimensional cradle sufficient to rest an end of a pipette.

The basin bed can include a resilient membrane comprised of a gas-permeable material and thin enough to allow oxygen and/or carbon dioxide permeation through the membrane.

The apparatus can further include protrusions projecting upward from the basin bed, the protrusions configured to protect biological cells between the protrusions. The protrusions can include rows of parallel fins with grooves therebetween configured to protect biological cells. The ledge can be located perpendicularly to the rows or at an end of the rows.

A height of the ledge can be within 25, 50, 75 100, 250, 500, 750, 1000, 1125, 1250, 1500, 1750, 2000, 2250, 2500 μm or more of the basin bed. If protrusions are present, the measurement may be either from the tops of the protrusions or the lowest bottoms around their bases.

The ledge can be referred to as a first ledge and the concave surround referred to as a first concave surround, and the apparatus can further include a second ledge set against or into the sidewall, the second ledge having a horizontal surface above the basin bed, and a second concave surround extending around a portion of the second ledge, the second ledge and the second surround forming a three-dimensional cradle sufficient to rest the end of the pipette. The second ledge can be located at an end of rows of parallel fins projecting upward from the basin bed. The first and second ledges can be located at different heights from each other. The first ledge can be directly above the second ledge. There can exist markings on the first and second ledges or the first and second surrounds, the markings indicating a height of each ledge from the basin bed, and/or a volume of liquid that the apparatus holds up to each ledge.

The basin bed can be circular with first and second ledges set into or against the internal surface of its sidewall, and the apparatus can further include a third ledge set into the sidewall, and a fourth ledge set into the sidewall, wherein the ledges are located at 90°to one another around a circumference of the basin bed. The ledges can be set into the sidewall an equal amount, and the amount and the circumference of the basin bed can form a substantially square right prism volume. The basin bed can be a polygon, and the first and second ledges can be in corners of the polygon.

A T-flask tray device can comprise the apparatus in which first and second ledges are set underneath one or more caps.

A multiwell culture plate can comprise 2, 3, 4, 6, 12, 24, 96, 384, or 1536 of the apparatuses that are integrally formed into a common molded polymer. The molded polymer can be an elastomer, and the multiwell culture plate can further include a rigid frame configured to mate with the elastomer and hold the basin beds of the apparatuses off of an underlying surface. The multiwell culture plate can further include a cover configured to mate with a top of the molded polymer, the cover having a plurality of drip seal projections configured to mate with the apparatuses in the molded polymer.

The surround can be entirely inset into the sidewall. A portion of the ledge can protrude from the sidewall to form a proscenium. The apparatus can further include a sharp corner between the surround and the sidewall or between the surround and the ledge. The horizontal surface of the ledge can be sloped toward or away from the basin bed.

Some embodiments are related to a method of manufacturing a cultureware vessel, the method including forming a basin bed, and molding a sidewall surrounding and sealed against the basin bed sufficient to hold liquid, a ledge set against or into the sidewall, the ledge having a horizontal surface above the basin bed, and a concave surround extending around a portion of the ledge, the ledge and the surround forming a three-dimensional cradle sufficient to rest an end of a pipette.

The forming can be in a gas-permeable material, the basin bed including a resilient membrane that is thin enough to allow oxygen permeation through the membrane. The method can further include fashioning protrusions that project upward from the basin bed, the protrusions configured to protect biological cells between the protrusions.

Some embodiments are related to a method of adding a ledge to a cultureware vessel, the method including providing a cultureware vessel having a basin bed and a sidewall surrounding and sealed against the basin bed sufficient to hold liquid, and setting a ledge against or into the sidewall, the ledge having a horizontal surface above the basin bed, and a concave surround extending around a portion of the ledge, the ledge and the surround forming a three-dimensional cradle sufficient to rest an end of a pipette.

Some embodiments are related to a cultureware vessel apparatus including a basin bed, a sidewall surrounding and sealed against the basin bed sufficient to hold liquid, a first ledge set against or into the sidewall, the first ledge set at a first height above the basin bed, and a second ledge set against or into the sidewall, the second ledge set at a second height above the basin bed, the second height being different than the first height.

The basin bed can include a resilient membrane comprised of a gas-permeable material and thin enough to allow oxygen permeation through the membrane. The apparatus can further include protrusions projecting upward from the basin bed, the protrusions configured to protect biological cells between the protrusions. The protrusions can include rows of parallel fins with grooves therebetween configured to protect the biological cells. The first ledge can be located perpendicular to the rows or at an end of the rows.

The first height or the second height can be within 25, 50, 75 100, 250, 500, 750, 1000, 1125, 1250, 1500, 1750, 2000, 2250, 2500 μm, or more of the basin bed.

The first and second ledges can share a common wall. The first and second ledges can be adjacent to one another. A third ledge can be set against or into an internal surface of the sidewall, the third ledge set at a third height above the basin bed, the third height being different than the first height and the second height. The first, second, and third ledges can be adjacent to one another.

Some embodiments are related to a method of manufacturing a cultureware vessel, the method including forming, such as by molding, a basin bed, and molding a sidewall surrounding and sealed against the basin bed sufficient to hold liquid, a first ledge set against or into the sidewall, the first ledge set at a first height above the basin bed, and a second ledge set against or into the sidewall, the second ledge set at a second height above the basin bed, the second height being different than the first height.

The forming can be in a gas-permeable material, the basin bed including a resilient membrane that is thin enough to allow diatomic oxygen or carbon dioxide permeation through the membrane. The forming can further include fashioning protrusions that project upward from the basin bed, the protrusions configured to protect biological cells between the protrusions.

Some embodiments are related to a method of adding ledges to a cultureware vessel, the method including providing a cultureware vessel with a basin bed and a sidewall surrounding and sealed against the basin bed sufficient to hold liquid, setting a first ledge against or into the sidewall, the first ledge set at a first height above the basin bed, and setting a second ledge against or into the sidewall, the second ledge set at a second height above the basin bed, the second height being different than the first height.

Some embodiments are related to a method of delicately adding liquid to a cultureware vessel, the method including resting a tip of a pipette on a ledge set against or into a sidewall of a cultureware vessel, the ledge having a horizontal surface above a basin bed of the cultureware vessel, dispensing liquid from the pipette onto the ledge, and allowing the liquid to fall from the ledge into the basin bed.

The tip of the pipette resting on the ledge can also abut a concave surround extending around a portion of the ledge. The method can include allowing the liquid to cascade over spillway protrusions under the ledge. A height of the ledge can be within 25, 50, 75 100, 250, 500, 750, 1000, 1125, 1250, 1500, 1750, 2000, 2250, 2500μm, or more of the basin bed. The liquid can be a culture media carrying biological seed cells.

Some embodiments are related to a method of adding a consistent height of liquid to a cultureware vessel, the method including dispensing a liquid into a cultureware vessel such that the liquid rises above a ledge set against or into a sidewall of the cultureware vessel, the ledge having a horizontal surface at a predetermined height above a basin bed of the cultureware vessel, resting a tip of a pipette on the ledge and underneath a top surface of the liquid, aspirating, using the pipette, the liquid from the cultureware, and stopping the aspirating when the top surface of the liquid sinks to a level equal with the ledge.

The stopping can occur because the top surface of the liquid sinks below at least a portion of a lumen in the tip of the pipette, thereby breaking seal with the liquid and preventing further aspiration. The pipette can project diagonally downward toward the ledge such that an entrance to a lumen in the tip projects upward. The tip of the pipette resting on the ledge can also abuts a concave surround extending around a portion of the ledge. The liquid can be a culture media, a phosphate buffered saline (PBS) solution, a washing solution, or an enzyme solution. A flow rate of the aspirated liquid can be below 0.5 milliliters (mL) per second.

The pipette can be referred to as a first pipette, and the method can further include resting a tip of a second pipette on a second ledge set against or into the internal sidewall of the cultureware, the second ledge having a different height above the bottom of the cultureware than the first ledge, wherein the dispensing is from the second pipette. The first pipette can be a Pasteur pipette or a micropipette, and the second pipette can be a micropipette or a serological pipette.

### BRIEF DESCRIPTION OF THE DRAWINGS

FIG. 1A is a top side perspective view of a cultureware vessel in accordance with an embodiment.

FIG. 1B is a plan view of the cultureware vessel of FIG. 1A.

FIG. 1C is a vertical cross-section perspective view of the cultureware vessel of FIG. 1A.

FIG. 2 is a top side perspective view of a cultureware vessel with squared ledge surrounds in accordance with an embodiment.

FIG. 3A is a top side perspective view of a 24-well plate in accordance with an embodiment.

FIG. 3B is a plan view of the 24-well plate of FIG. 3A.

FIG. 4 illustrates a cover configured to mate with a 24-well plate in accordance with an embodiment.

FIG. 5A is a top side perspective view of a single-well plate in accordance with an embodiment.

FIG. 5B is a plan view of the single-well plate of FIG. 5A.

FIG. 5C is a cross-section plan view of the single-well plate of FIG. 5A.

FIG. 6 is a top side view of a petri dish in accordance with an embodiment.

FIG. 7 is a top side view of a petri dish with stepped side ledges in accordance with an embodiment.

FIG. 8 is a top side perspective view of a T-flask tray in accordance with an embodiment.

FIG. 9 illustrates a ledge with an engraved spillway in accordance with an embodiment.

FIG. 10 illustrates a ledge with a lateral stepped spillway in accordance with an embodiment.

FIG. 11 illustrates a ledge with a binomial spillway in accordance with an embodiment.

FIG. 12 illustrates a ledge with a protruding weir spillway in accordance with an embodiment.

FIG. 13 illustrates a ledge with a horizontal shelf spillway in accordance with an embodiment.

FIG. 14 illustrates a ledge in an inside corner in accordance with an embodiment.

FIG. 15 is a flowchart illustrating a process in accordance with an embodiment.

FIG. 16 is a flowchart illustrating a process in accordance with an embodiment.

FIG. 17 is a flowchart illustrating a process in accordance with an embodiment.

FIG. 18 is a flowchart illustrating a process in accordance with an embodiment.

### DETAILED DESCRIPTION

Generally, cultureware is described that has ledges built in to its inner walls that are sized and positioned for a user to rest the end of a pipette while filling or aspirating. If the bottom of the cultureware dish is made of thin and delicate material, the ledge presents a hardened area that resists puncture and is therefore a preferred area for the relatively sharp pipette end to rest. If the bottom of the cultureware has directional features, such as elongated protrusions, the ledge may be positioned perpendicular or parallel to (or somewhere between) the directional features so that material being distributed to or aspirated from the cultureware is more protected or more exposed.

The ledge's height above the bottom of the cultureware can be predetermined for a specified depth or volume of liquid. That depth or volume can be marked near the ledge. Filling the cultureware's basin and then aspirating from a pipette's tip resting on the ledge can ensure that the amount of liquid left in the basin is accurate, or at least repeatedly precise as compared with other methods.

A “shelf,” “landing,” “ledge,” “platform,” or “weir” are terms that are referred to interchangeably and describe a relatively horizontal surface against or within a wall that is elevated above another surface, or as otherwise known in the art. A weir additionally includes a low wall.

A “brink” or “crest” is the top of a waterfall, or as otherwise known in the art.

A “cirque” is a half-open steep sided hollow in a mountain or other mass, or as otherwise known in the art.

A “gorge” or “glen” is a deep narrow valley with steep walls, or as otherwise known in the art.

A “hanging valley” is an elevated valley, or as otherwise known in the art.

A “spillway” includes an area underneath a ledge that is engineered with partial obstructions to remove kinetic energy from falling liquid by interrupting its fall to roil the liquid, or as otherwise known in the art.

A “tiered waterfall” is a waterfall with multiple landings, or as otherwise known in the art.

A ledge can exist on the inside wall of a piece of cultureware to divert flow from a pipette laterally (i.e., in the horizontal plane) over directional grooves/niches/lanes in the bottom. One or more ledges can extend along an entire wall length of dish ware. The ledge can be perpendicular to the niches or lanes in a gas permeable membrane in order to shield cells within them from flow and shear from liquid dispensed from the ledge.

A ledge can be radiused or chamfered around its edges or corners to convert vertical flow efficiently into horizontal flow. This can facilitate cell removal from the device. A radiused or chamfered edge may be used to redirect fluid, such as from a pipette, in a horizontal manner, parallel to the orientation of fins, to assist with bubble removal during media filling and to assist with cell removal during harvesting. By incorporating radiused edges on opposite sides of the dish, a half pipe structure can be formed. This can facilitate the user in dislodging bubbles or cells by performing a sloshing or tilting action in a direction parallel with the fins. Fin edges can be rounded, pointed, or non-blunt to mitigate splashing of fluid that is incident on them.

A shelf can delimit the maximum fill height of a vessel. This can help in practical experiments because, otherwise, there may be no other limit, such as oxygen transport to cells, to filling a gas permeable device. In an open gas permeable device there is a risk of overfilling, which can lead to spilling from sloshing or handling. Excess fluid above the shelf can be removed by resting a pipette against the maximum fill shelf and aspirating all fluid above that point. The ledge can act as a weir or as an indicator.

Ledges with heights in between set maximum and minimum heights can provide distinct levels to feed cells a quantifiable and repeatable amount of media per feeding (i.e. a media exchange). This can be used to determine consumption over time and therefore calculate feeding parameters for subsequent use in related perfusion system. In addition, the height of the shelves can be set to allow the addition of standardized amounts of reagents, supplements, etc. for biological assays or in vitro/in situ tests. Plates can come with ledges for one or multiple such supplements. The ledges may be designed in a helical or perimeter stepped staircase around device to provide many gradations.

In accuracy and precision tests with 24-well devices in which each well was filled to the brim with 5 mL of cell culture water, an experimenter was able to aspirate down to a nominal 4.5 mL shelf to 4.52 mL with a tolerance of ±0.074 mL. This is within 1.8% of the nominal volume. Similarly, he was able to aspirate down to a nominal 3 mL shelf to 3.06 mL±0.047 mL, which is within 3.6%. For a nominal 1 mL shelf, the result was 1.05 mL±0.039 mL (9%). For a nominal 0.25 mL shelf, the result was 0.25 mL±0.019 mL (7%).

A technical advantage of aspirating to shelf heights is that media volumes can be precisely dispensed from one large (e.g., 25 mL, 50 mL, 100 mL) serological pipette into all wells of a multi-well plate rather than needing to individually meter from a precision pipette into each well.

A total number of shelves in a particular vessel is not limited to 1, 2, 3, 4, or any particular number. There can be fewer or more shelves, especially in larger plates. In addition, shelves can be placed with mirrored symmetry to accommodate left-handed and right-handed human or robotic operators.

The shelves can be placed at specific angles and orientations relative to a patterned membrane, such as perpendicular or parallel to a groove geometry. In a perpendicular orientation, the shelves can assist media exchange without disturbing cells that are lodged within the grooves. Ledges for this can have minimum height for removing the media and a maximum height shelf for adding media. In a parallel orientation, the ledges can be used to assist fluid flow along the grooves removing air when wetting, or cells during collection. That is, fluid flow running parallel down the grooves may be more laminar and reach the full extent of each groove's cross section with its ram pressure.

Regions of the gas permeable membrane can be compartmentalized by addition of a divider, for example a wall, half-wall, septum, or fence. Compartments can allow for independent seeding, sampling, recovery, or monitoring within the vessel. Physical separations can be accomplished by segmenting a single or multiple lanes/niches longitudinally, segmenting a patch or region with a low fence within the membrane, or segmenting a corner or edge portion of the device. This region can also be used as an initial cell culture space within a larger membrane such that a low cell population can be grown from an optimal seeding density and then dispersed out over the full membrane for further expansion. This can accommodate situations where the seeding density would be too low otherwise.

A technical advantage of this is that it may obviate the need to do a preculture in a smaller vessel and transfer to the production vessel, i.e., a step in a seed train. The compartmentalized region can also have its own set of ledges to facilitate operations such as feeding, staining, enzymatic digestion/trypsanizing, harvesting, or conducting assays. Multiple compartmentalized regions may be laid out across the membrane or in an array-like structure.

Ledges can be made lower than the membrane to allow for full extraction of biological or other material, such as a sump point.

Clearly marked, ledges can give a safe, easily identifiable place to place a pipette away from the delicate membrane to prevent puncture. This is useful for both human and robotic operators. In other words, the shelves can act as a landing pad with thicker or more robust material.

Ledges can be designed to spread the entry of media into the vessel. In order to reduce fluid velocity and turbulence it is beneficial to disburse the fluid flow from the stream coming out of a pipette, tubing, or container out over a greater length. This can be achieved by creating a shelf with a retrograde profile or adding a lip (weir) to the shelf, such that fluid spreads out then waterfalls over across the length of the shelf. Castellations may be added along the shelf to control the flow. A singular large shelf with a weir like patterning may also be incorporated to allow for large volumes of liquids to be easily poured into the device and evenly dispersed into the overall device. Drains or holes can be placed in the shelf to control the flow of fluid. A portion of the shelf may be angled to distribute the flow across a length of the device. The shelving may be stepped (e.g. like a stepped spillway). Providing multiple steps that the fluid cascades down helps reduce fluid velocity, distribute fluid, and reduce large turbulence structures. Grooves or engravings can provide capillary paths to disperse flow fields or direct it in a given direction to minimize turbulence. Shelves may be designed in a helical or perimeter stepped staircase around device to minimize fluid velocity, turbulence, and direct the fluid to fill from a given orientation.

A reservoir greater than the gas permeable membrane area can provide sufficient media or waste buffering capacity for high density cell cultures (e.g. trapezoidal, stepped pyramidal, partitioned area within reservoir). This allows for increased periods between feedings and a shallower media height per volume. The result is a vessel that is more compatible for camera imaging of a cell cultures (i.e., liquid media absorbs light, so too tall of a column makes the image too dark), and provides a more favorable approach angle for pipetting operations.

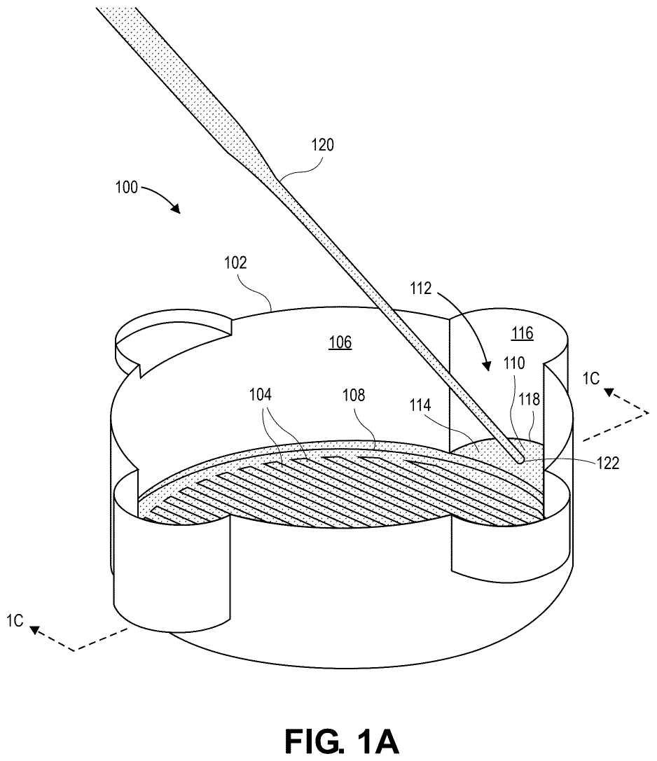

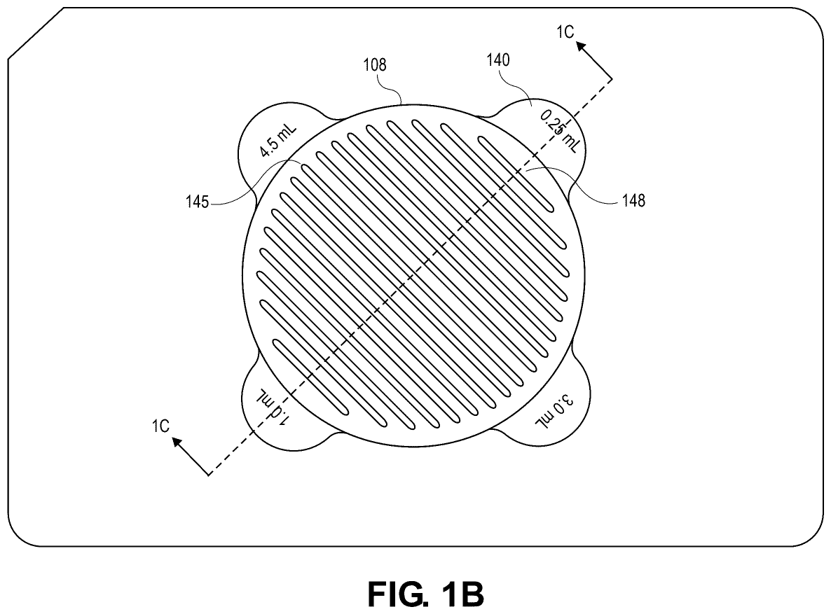

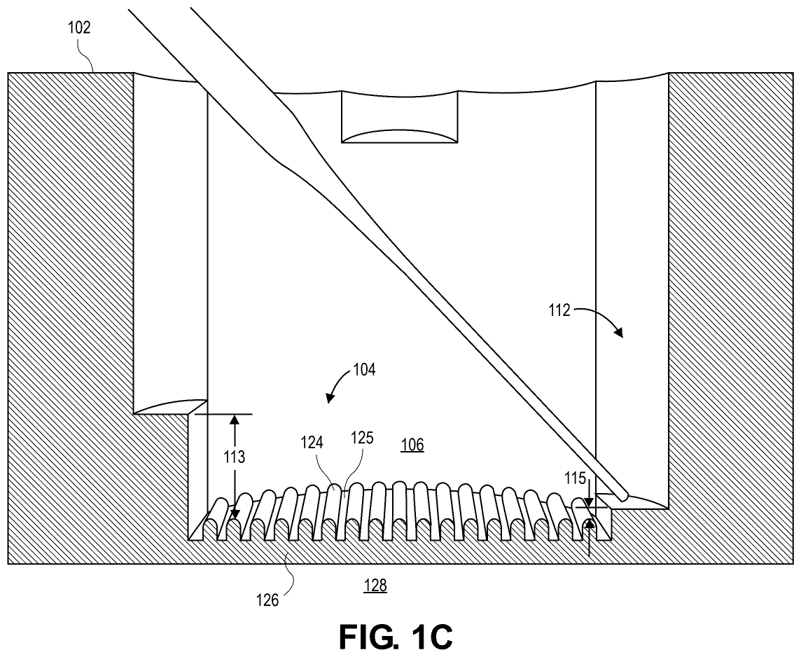

FIGS. 1A-1C illustrate system 100 in which thin Pasteur pipette 120 rests in cultureware vessel 102. Cultureware vessel 102 includes sidewall 106 surrounding and hermetically sealed against basin bed 108. Thus, the cultureware vessel is able to hold and store liquids.

Within sidewall 106 is ledge 112. Forming the bottom, base of ledge 112 is horizontal surface 114. Cupping around the ledge vertically is surround 116, which joins horizontal surface 114 at inside edge 118. Horizontal surface 114 and surround 116 form three-dimensional (3D) cradle 110.

Cradle 110 is said to capture distal end 122 of pipette 120. Even though pipette end 122 does not rest against inside edge 118, the pipette end is surrounded in a concave space that prevents it from escaping uncontrollably. For example, if a person holding the pipette accidentally knocks it forward, distal end 122 would likely side over horizontal surface 114 into edge 118 and get pinned into edge 118—going no further. Small side-to-side mis-directions would not result in distal end 112 getting away or leaving the safety of ledge 112.

A technical advantage of the 3D cradle is in keeping the relatively sharp end of the pipette away from the bottom of the cultureware. Basin bed 108 is formed of a thin membrane that is easily poked through. In turn, rows of surface features 104 in the thin membrane protect delicate biological cells between them and provide oxygen due to their permeability. Keeping the pipette secured away from the basin bed lessens the risk of puncture.

A “horizontal surface” does not need to be flat but can be sloped toward or away from the basin bed. The slope may be 0.5, 1, 2, 3, 4, 5, 6, 7, 8, 9, 10, 12.5, 15, 17.5, 20, 22.5, 25, 27.5, 30, 35, 40, 45 degrees, or other tilt angles. Water typically needs a 1-2% grade to drain from large outdoor surfaces. A slope toward the basin bed may help fluid drain from the ledge into the basin bed. A slope away from the basin bed can be called a retrograde slope. The horizontal surface should be compatible with the cradle or other aspects of the design.

FIG. 1B is a top-down view that illustrates the ledges at 90° intervals from one another around the circumference of basin bed 108. Ledges can be spread apart at equal angles, like those shown, or bunched along a certain slice. Ledges around a basin can be all at the same height or at different heights. Ledges at different heights can be directly on top of each other or staggered.

Markings 140 on the ledge horizontal surfaces show the volume of liquid that is held in the vessel up to the horizontal surface, like a gradation. Different markings are shown on the different ledges, which are at different heights above the basin bed. The markings can be shown on the surrounds or adjacent each ledge for clear readability.

Different markings are shown for different ledges. The tallest ledge can be used as a fill line for the top media level in the well. Shorter ledges can be used for harvesting and flushing, or however an operator may determine. The shortest ledge may have a horizontal surface (ledge height) that is lower than, even with, or higher than the tops of the fins.

In the exemplar, the ledge height is within 800 μm of the tops of the fins, which are 500 μm tall, to be a total of 1300 μm from the bottom of the grooves.

A technical advantage in physical shelves is in that they avoid the error introduced by parallax when operators try to fill to printed or etched fill lines or gradations. They obviate the need for holding device up to one's eye, or bringing one's eye to the media height for observation, somewhat impractical for interior wells. Filling above and then aspirating to the shelf height provides a method that is relatively independent of operator judgement and technique, which increases reproducibility.

Meniscus effects can affect the precise volumes defined by the shelves. Treatment of the media with a surfactant or surface tension-reducing additive can mitigate meniscus effects. Forming the vessel wall and shelves with materials that are neither very hydrophilic or very hydrophobic (e.g. having around a 90-degree contact angle or amphiphilic) can also mitigate meniscus effects.

Sloping the shelves towards the interior of the vessel can to prevent cells or other solids from settling on the shelves during seeding. Alternatively, or in addition, slots or grooves that slope towards the interior of the vessel can be used to prevent cells from settling in the shelf during seeding but also provide a more stable location to rest a pipette. The pipette contacting surface can then be horizontal or retrograde in angle. Surfaces should sometimes be radiused or angled to prevent cell settling. Alternatively, the shelf may be retrograde.

Ledges may be colored to ease visualization. Shelves may also be textured to provide tactile feedback to user and prevent slippage of pipettes on the shelf surface.

Assessing the level of the culture vessel can be crucial given that deviations can lead to the accumulation of biomass at the low area and deficit of biomass at the high areas leading to non-uniform device performance. This is particularly important for flat bottomed gas permeable devices where deviations from true level can greatly affect biological performance as they are predicated on cells being given distances from the oxygenating surfaces. It is advantageous to place shelves of known height at multiple places around the dish to ensure level, or engraving level rings or perimeter marking at various heights for the purposes of leveling. By setting the markings at the same height as the shelves, the user can more accurately determine level as they do not need to interpolate between level rings.

The figure shows end 145 of rows that is parallel with the rows. The ledge at end 145 or its opposite can be used to pipette liquid for flushing liquid along the rows and through the gaps.

The figure also shows end 148 that is perpendicular with the rows. The ledge at end 148 or its opposite can be used to pipette liquid in a careful manner so as to not disturb cells that are within the rows.

FIG. 1C shows a cross section of two ledges, one at taller height 113 and one at lower height 115. A third ledge is in the top background. In the exemplar, height is measured against a top of surface features in the basin bed.

Surface features 104 include fins 124 that protrude upward from the basin bed and longitudinally extend into and out of the page. Aligned with and between fins 124 are grooves 125 in which cells or other microorganisms may grow.

Thin membrane 126 allows oxygen to permeate from underside 128, through the bulk membrane, into fins 124, and out to grooves 125. Waste gases, such as carbon dioxide, can permeate in an opposite direction.

Taller height 113 ledge may be used for adding or replacing media. Lower height 115 ledge may be used for harvesting or, at the initial stage, seeding of cells in the grooves. The particular ledge's use depends on best practices and operator preference.

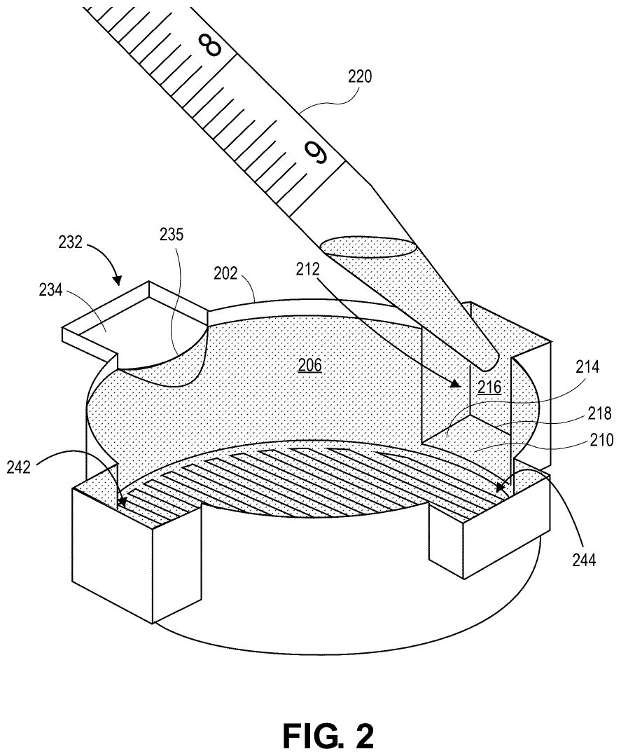

FIG. 2 shows a cultureware vessel with squared ledge surrounds. Cultureware vessel 202 includes four ledges set into sidewall 206: ledges 212, 232, 242, and 244.

Like ledge 112 in FIG. 1, ledge 212 in FIG. 2 is set into a sidewall. Ledge 212 is formed from horizontal surface 214 meeting surround 216 at edge 218, the combination of which form cradle 210. Unlike ledge 112, concave ledge 212 has a surround with three flat, rectangular walls that meet at sharp corners. Surround 216 is not curved but rather has a rectangular planform where it meets its floor.

Besides rectangular embodiments, the planform of surrounds may be polygonal, having any number of flat walls meeting at corners that are obtuse, acute, or 90°.

Cradle 210 encircles the end of serological pipette 220, which is positioned at a 45° angle into it. Even though the tip of the serological pipette is shown raised off of horizontal surface 214, large movements of the tip would strike horizontal surface 214, surround 216, or the intersection of them at edge 218 and thus be contained. In this way, the pipette's end is cradled.

Ledge 232, which sits 90° counterclockwise from ledge 212 on sidewall 206, includes proscenium 235. The proscenium is the portion of horizontal surface 234 that protrudes out from sidewall 206. Proscenium 235 bows out from the sidewall in a curved manner and has an underside that gradually mates with the sidewall underneath.

A technical advantage of a proscenium is a larger pad resting in which to place the end of a pipette and thus more protection for the bottom of the vessel.

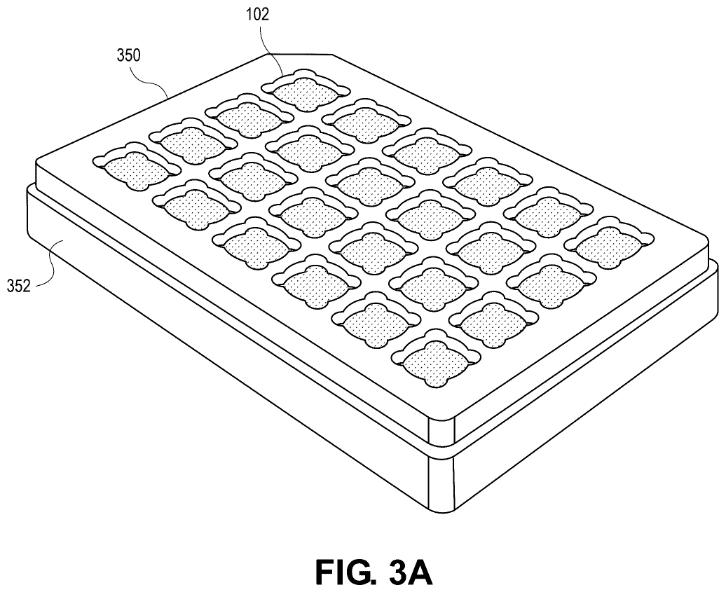

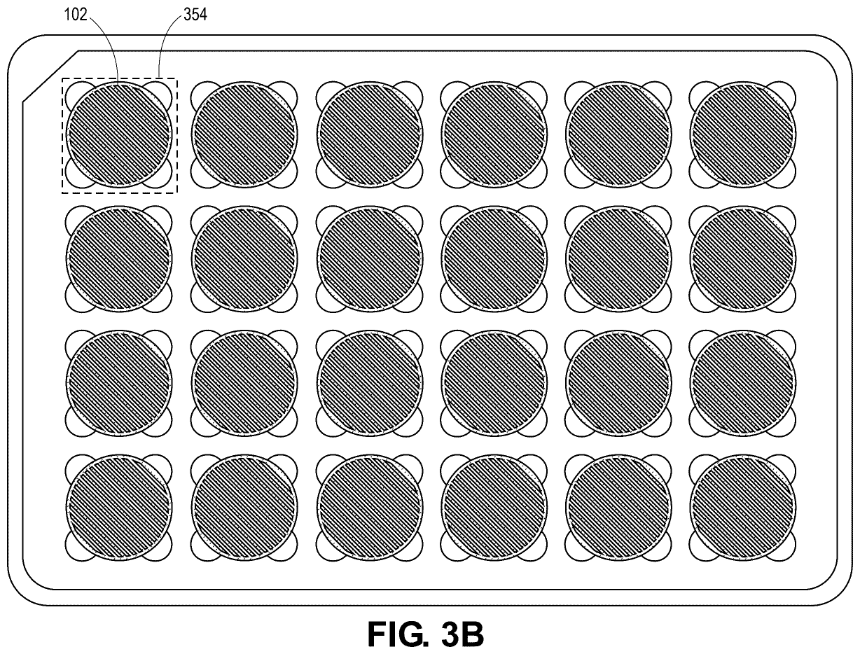

FIGS. 3A-3B show 24-well plate 350 with twenty-four cultureware vessels 102, each vessel having four equally spaced ledges. The well plate, its vessels and ledges, and basin beds are comprised of a common, integrally formed elastomer. In this case, the elastomer is a resilient silicone polymer.

The resilient silicone polymer is thin enough in the basin beds to allow diatomic oxygen and other gases to permeate therethrough. Because the silicone polymer is resilient, the ledges and sides are somewhat resilient as well—as well as the entire well plate.

In order to keep the resilient well plate from sagging and to keep dimensionality of the internal wells when it is handled or centrifuged, resilient well plate 350 is mated into rigid frame 352. Rigid frame 352 incorporates circular holes in its bottom under each of the twenty four wells. Small standoff feet protrude downward from the resilient basin bed. The feet stand against a flat base of the frame. The frame includes slots in order to provide airflow between the flat bottom and the silicone membrane. This allows airflow underneath so that gas can reach the undersides of the membranes. The silicone feet also help support the membranes during centrifuging.

FIG. 3B shows a top-down view in which the circular ledges protrude into their respective sidewalls at 90-degree intervals. The circular ledges protrude an equal amount into the sidewall and enough to fill in most of the “corner” areas in the bulk material. This combination of simple shapes makes somewhat of a square. The basin and ledges, being three-dimensional, make a substantially square right prism volume 354 in the bulk material. Twenty-four of the square volumes can coexist in the well plate without expanding the well plate's size from standard such well plates.

FIG. 4 illustrates cover 400 that is configured to mate with the molded polymer 24-well plate of FIG. 3A. Cover 400 includes circular projections 401 that mate with the inner wall of each well. The ring projections gather condensate and, through the benefit of their relatively sharp ends, drip the condensate back into its respective well.

Cover 400 is made of clear polystyrene and has a corner cut out to mate with a respective corner in the well plate. Covers can be made in the shape of their mating vessels and include or not include features to help condensate find its way back into its respective well.

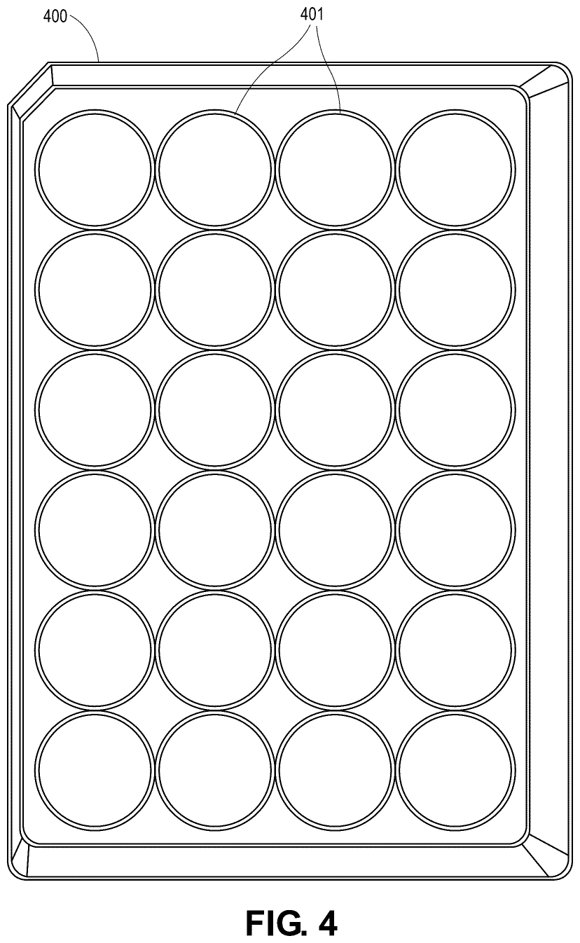

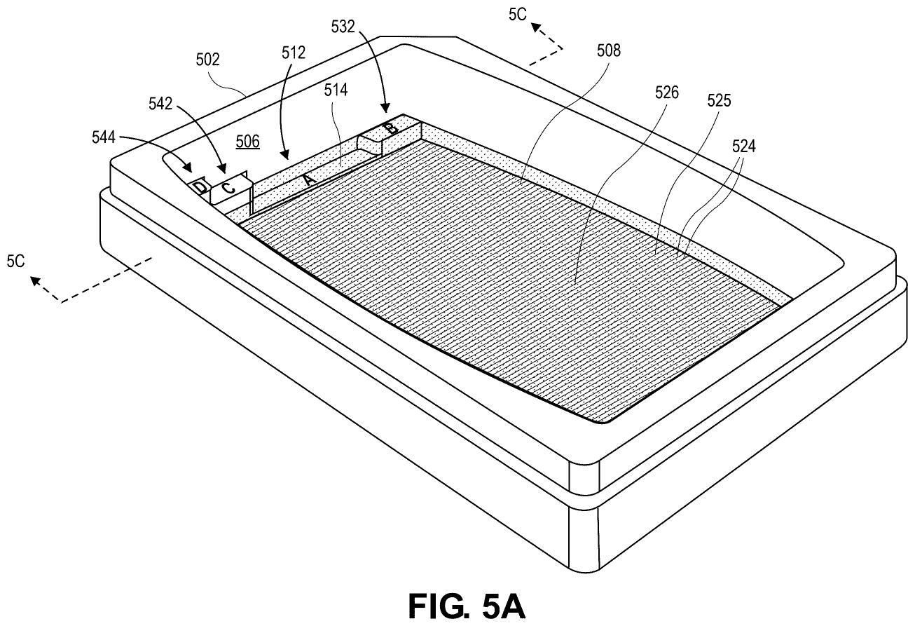

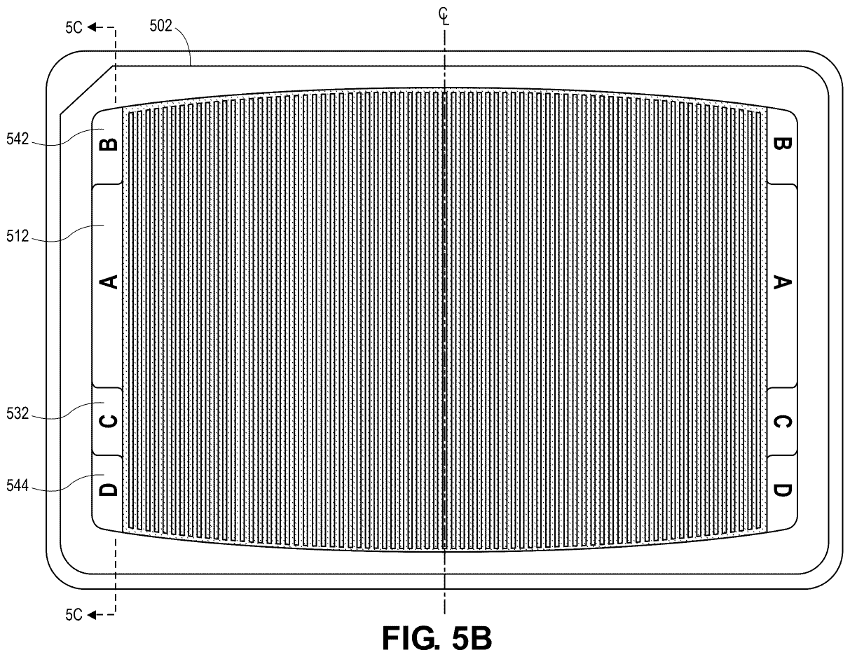

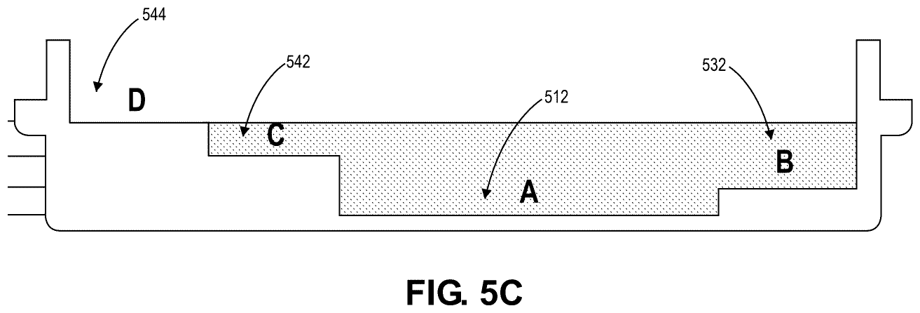

FIGS. 5A-5C illustrate a single-well plate cultureware vessel 502. In vessel 502, sidewall 506 surrounds basin bed 508 in a straight and slightly rounded configuration, like a bathtub.

Basin bed 526 is a resilient polymer membrane with upward protrusions 524. The membrane and protrusions are gas permeable, thin enough to allow gas to permeate from an underside of the membrane to the topside of it. Protrusions 524 create groove niches 525 therebetween, where cells can grow and be protected from the shear forces caused by large movements of liquid media.

Along the left end of the interior of vessel 502 are four ledges set against sidewall 506. The ledges are ledge 512, 532, 542, and 544. All four share a common flat wall of sidewall 506.

As shown in FIGS. 5A and 5C, ledge 512 is at a lowest height above basin bed 508. That is, horizontal surface 514 of ledge 512 is only slightly above the basin bed, in particular about 500 μm above the basin bed. Meanwhile, the horizontal surface of ledge 532 is higher than first ledge 512. Those of ledges 542 and 544 are higher still. The assemblage resembles an inverted Olympic podium with four levels.

Being lower and between the other ledges, ledge 512 and sidewall 506 form a cradle that can prevent the tip of a pipette from wandering too far astray. Ledge 532 forms a cradle with the two facets and corner of sidewall 506, and so does highest ledge 544. Ledge 542 forms a cradle in a corner where its horizontal surface, sidewall 506, and the wall below ledge 544 meet.

Ledge 512 is wider than the other ledges. In some embodiments, it can incorporate horizontal surface features to improve liquid distribution, drainage, or aspiration.

FIG. 5B shows ledges 512, 532, 542, and 544 mirrored at an opposite end of single-well vessel 502. A technical advantage of including a second set of ledges is that right-and left-handed users may be better accommodated. Further, the vessel can be better leveled if liquid rises to the same height with respect to matching shelves at each end.

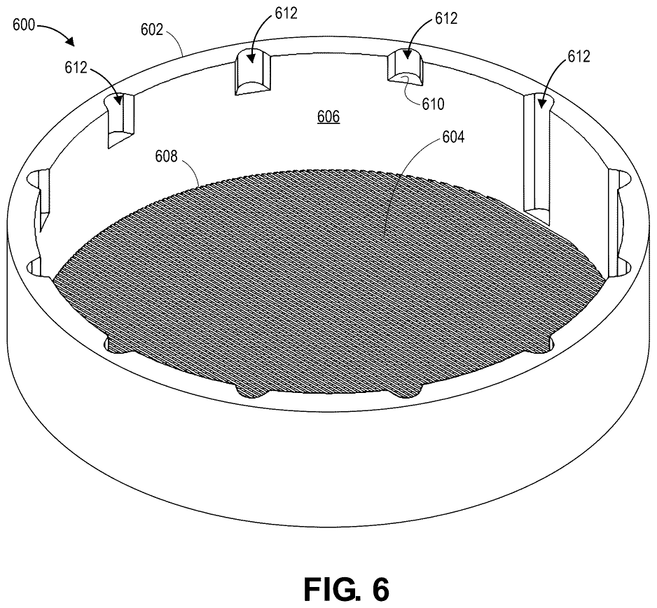

FIG. 6 is a top side perspective view of petri dish 600 in the form of circular cultureware vessel 602. The petri dish includes basin bottom 608 with integrated surface features 604 projecting upward. The basin is surrounded by sidewall 606.

Within sidewall 606 are ledges 612, each of which has a horizontal surface and a curved surround. The horizontal surface and surround form cradle 610 in which the tip of a pipette could be rested.

Twelve ledges 612 are set around the inner circumference of petri dish 600 at equal 30° intervals. The ledges are identical—except that they are at different heights from one another. The heights of the ledges progress from the lowest, which is on the right of the figure, clockwise around the circumference.

In other embodiments, different heights can be interspersed with one another or follow other patterns around the circumference or perimeter of the vessel. Ledges can be at different widths, shapes, tip-resting areas, horizontal surface features, and horizontal surface angles.

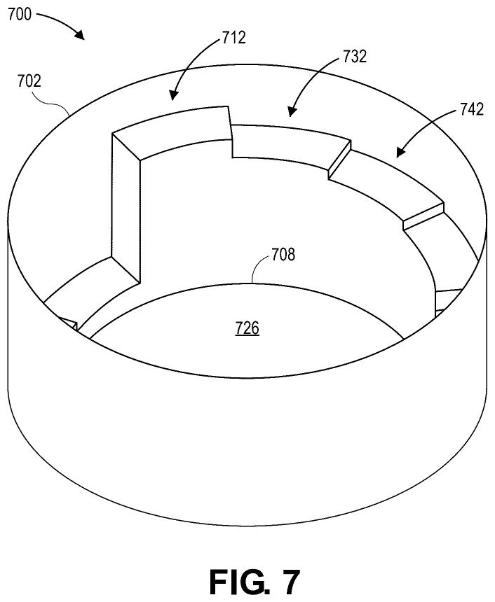

FIG. 7 is a top side perspective view of a petri dish 700 in the form of circular cultureware vessel 702. The petri dish has basin bottom 708, which is formed of thin membrane 726.

Surrounding basin bottom 708 are stepped side ledges 712, 732, 742, and others. The ledges are set against the sidewall, using the sidewall as their back. The ledges progressing deeper and deeper (i.e., in descending height) in a staircase-like fashion around the inner circumference of vessel 702.

In other embodiments, the ledge heights can be interspersed with one another or follow different patterns. The material beneath the higher ledges can give way to more space underneath for the basin bottom.

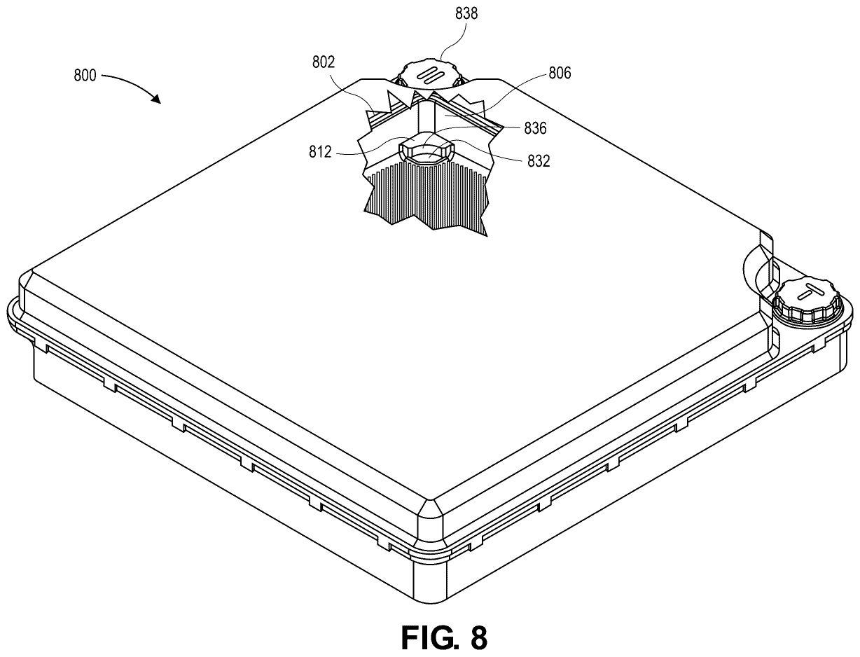

FIG. 8 is a top side perspective view of T-flask tray 800 in the form of closed cultureware vessel 802 with a cutaway to see its internal corner. Within vessel tray 802 is sidewall 806 that surrounds a bottom basin. Sidewall 806 wraps around a corner of the basin underneath cap 838. The cap is marked to indicate that its entrance is parallel with elongated ribs on the bottom.

Under the corner is ledge 812, with a horizontal surface that forms a cradle with the sidewall corner. Immediately below ledge 812 is another ledge.

Ledge 832 is immediately below ledge 812, set against its supporting wall 836. Supporting wall 836 extends vertically from lower ledge 832 to higher ledge 812. Liquid that is deposited onto ledge 812 can waterfall down supporting wall 836 onto ledge 832 before making its way to the basin bottom.

A technical advantage of a ledge being directly above another ledge is that both ledges may share a common opening and share a common flowpath, such as that provided by cap 838. The lower ledge can break the hydraulic ram force of liquid descending from the higher ledge. The functions of the two ledges can be clearly delineated, as it is easier for a human operator to see which is the higher and which is the lower ledge.

There may exist labelling on the lid or cap to indicate which shelves are at what height or serve what purpose. As shown, caps can show which opening is for parallel flow and which is for perpendicular flow.

For closed devices, a tube or a bulkhead fitting can be positioned at the various shelves to provide the ability to add or remove fluid and biological material. Such a tubing or bulkhead may be designed to be mobile to access multiple locations within the device, for example by pivoting, flexible connection, means of actuation. The feedthrough tubing or pipette can rest on shelving designing to accommodate angular motions to set fill or aspiration height.

The shelfing in a multi-well plate can facilitate the removal and addition of media in a controlled manner. This can help generate growth curves by counting a subset of growth in wells each day. Growth curves for different feeding strategies can be compared to determine the most optimal strategy (e.g. based on cost per cell or bioproduct yield, yield per device/area/volume, yield per time). For example, cells can be seeded into the wells with groups of wells receiving different amounts of media in a bolus feeding strategy. Cell counts after a given period of time can be measured. Fold-change in cell numbers or bioproduct produced can be used to determine a specific yield per amount of media. In an alternative method, cells are seeded into a multi-well device and groups are fed with different amounts of media per unit time. Cell or bioproduct yields are measured, and an optimal feeding rate is determined. Data from all of these methods can be used to infer an equivalent perfusion rate for adapting the bioproduction strategy to a perfusion bioreactor.

The shelf architecture can be made to be compatible with standard means of transferring fluid, including the use of pipettes (e.g. serological, graduated, micropipette, Pasteur, transfer, dropper, single channel, multichannel, repeater), the use of tubing (e.g. flexible, rigid, silicone, glass, plastic, polyethylene, polypropylene, polyvinyl choride (PVC), polytetrafluoroethylene (PTFE), TYGON® tubing, rubber, vacuum, high-performance liquid chromatography (HPLC), peristaltic, capillary, weldable), fluidic connectors (e.g. sterile connects and disconnects, pressfits, compression fittings, push-to-connect, threaded fittings, hosebarbs, Luer fittings, quick disconnect couplings, thermally or chemically welded fittings, bulkhead or throughwall fittings), or pumps (e.g. pipette controller, electronic pipette controller, peristaltic pump, vacuum pump, diaphragm pump, positive displacement pumps, centrifugal pumps, venturi pump, gravity feeding, siphons) or a combination thereof. Fluid may be pushed into the system (e.g. via pumping), pulled into the system (e.g. through vacuum), or poured into the system; fluid may be pushed out of the system (e.g. via pressurization with gas), pulled out of the system (e.g. through vacuum), or poured out of the system.

Some embodiments relate to the use of shelf-containing plates, dishes, trays, and devices to properly expand cells in vitro with minimal cellular disruption for:
a. Production of endosomes, extracellular vesicles (EVs), microvesicles, biological nanoparticles, lipid bilayers, therapeutic vesicles, nanovesicles, cell-derived vesicles;
b. Production of TIL (tumor-infiltrating lymphocyte) therapies, adoptive cell therapies, tumor-reactive lymphocytes, autologous T cell therapies, personalized immunotherapies, T cell receptor therapies, chimeric antigen receptor (CAR)-T therapies, regulatory T-cell therapies, T cell therapies, NK cell therapies, dendritic cell therapies, macrophage-based therapies, and γδ T-cell therapies; allogeneic cell-based therapies;
c. Expansion of cells in vitro as organoids; expansion of cells in vitro to be used for in vivo studies, such as cancer cells for tumor growth and tumor growth inhibition studies;
expansion of immune cells for in vitro and in vivo studies; expansion of primary cells such as fibroblasts for in vitro and in vivo studies;
d. Stem cell therapies that use hematopoietic, bone marrow, cord blood transplant, mesenchymal, induced pluripotent, neural, cardiac, epidermal, embryonic, organoid-based, or dental pulp stem cells; and cancer stem cells expanded for anti-cancer drug discovery.
e. Therapies that consist of pancreatic beta-cells, hepatocytes, myoblasts, fibroblasts, olfactory ensheathing cells, Schwann cells, retinal pigment epithelium cells, islet cells, and adipocytes.
f. Production of biological products, such as proteins and viruses, using the following cell lines: Chinese hamster ovary (CHO), human embryonic kidney (HEK)293, Spodoptera frugiperda (SF)9, A549 adenocarcinomic human alveolar basal epithelial cells, Henrietta Lacks (HeLa), Vero African green monkey, baby hamster kidney (BHK), human cell line PER. C6, Madin-Darby canine kidney (MDCK), hybridomas, bacterial cells, yeast cells, plant cells, fungal cells, insect cells and immortalized cell lines.

Some embodiments relate to the use of shelf-containing and gas-permeable membrane-containing dishes, tray, plates, and devices for providing precise atmospheric condition in vitro to expand stem cells, while retaining stemness. Devices can be operated under controlled atmospheres including hyperoxic, normoxic, physiologic, hypoxic, and anoxic conditions. For example, oxygen tension can be an important parameter for the culture of stem cells. Gas permeable devices in combination with an oxygen-controlled incubator or hypoxic chamber can be used to expand stem cells under defined conditions while maintain phenotypic plasticity.

Some embodiments relate to the use of shelf-containing and gas-permeable membrane-containing dishes, tray, plates, and devices for providing precise atmospheric conditions to expand T-cell therapies in vitro, while maintaining differentiation and t-cell memory phenotypes.

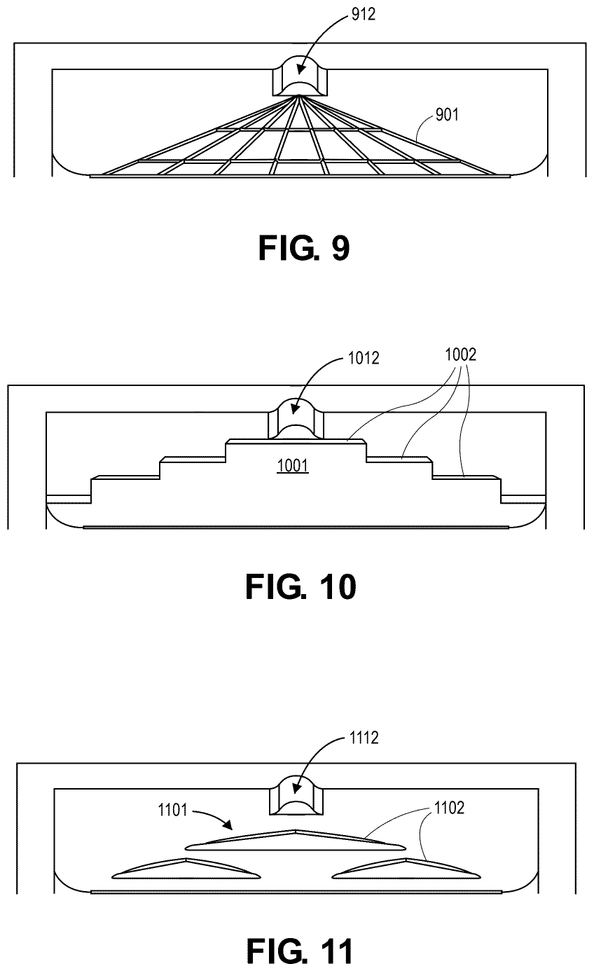

FIG. 9 illustrates ledge 912 with engraved spillway 901. Engraved spillway includes a diverging web of relief features over which liquid may dribble. The relief features spread apart the liquid from droplets into more of a flat sheet that cascades down to the bottom basin or lower shelves.

FIG. 10 illustrates ledge 1012 with lateral stepped spillway 1001. Lateral stepped spillway 1001 includes horizontal surfaces 1002 that first bifurcate then step down the fluid to lower levels. The potential energy of the liquid, which turns to kinetic energy as it falls, is lost to small amounts of roiling at the base of each step as it waterfalls down.

FIG. 11 illustrates ledge 1112 with binomial spillway 1101. Obtuse triangular protrusions with slanted horizontal surfaces 1102 continuously bifurcate the fluid into smaller and smaller volumes. As pictured in the embodiment, liquid pipetted onto the ledge would be split into four different parts by the time it gets to the bottom of the embodiment.

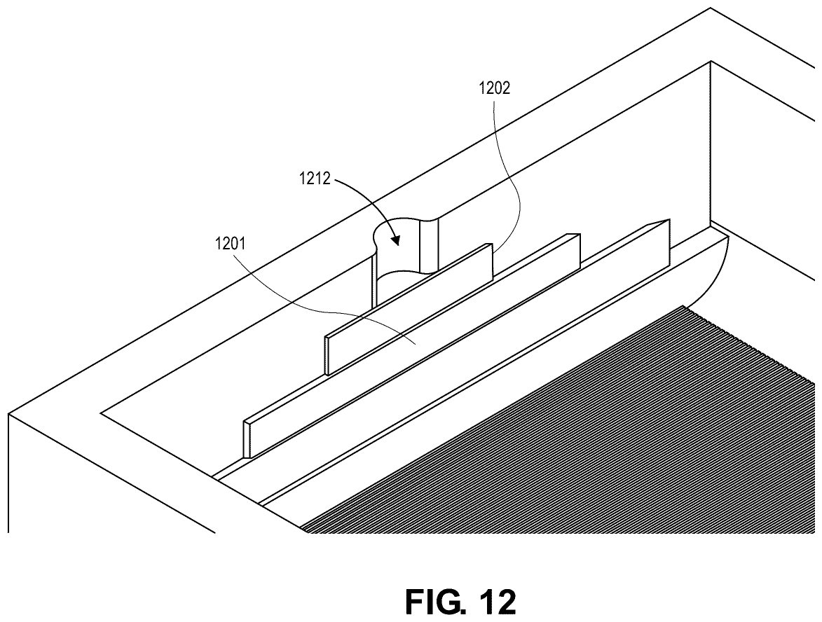

FIG. 12 illustrates ledge 1212 with protruding weir spillway 1201. Low wall weirs 1202 hold back the liquid on ledge 1212 until it spills over it onto other protruding rectangular features. The spillway features multiple rectangular projections, coursing all of the way down to the bottom basin. Note that the sidewall at the same level as the lowest projection is rounded.

FIG. 13 is a cross section of ledge 1302 and shows an upper side perspective view of horizontal shelf spillway 1301. Horizontal shelf spillway includes upward turned, French cleat-like protrusions. Unlike a gutter, each protrusion allows a small amount of liquid to fill its topside before its overflows and cascades liquid over its top to a protrusion or basin bed below.

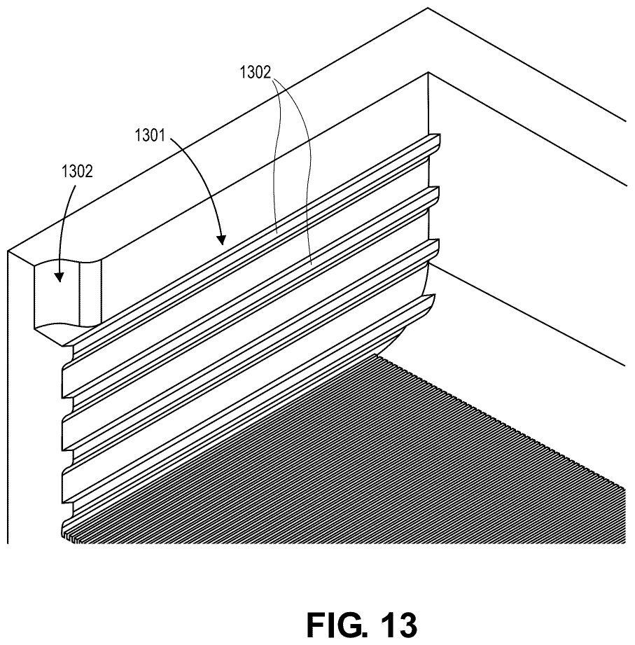

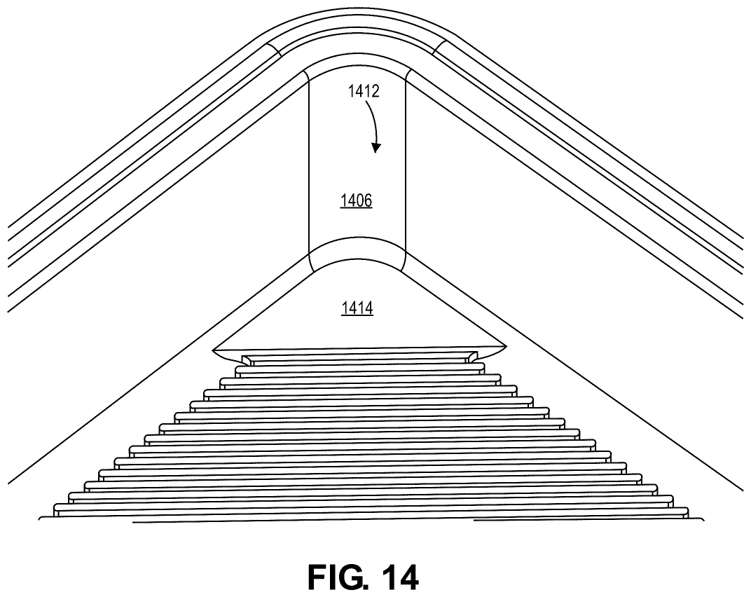

FIG. 14 illustrates ledge 1412 up against a rounded internal corner. The rounded corner is part of sidewall 1406 that span around to each side. Below the corner is horizontal surface 1414. Horizontal surface 1414 and the corner formed by sidewall 1406 forms a cradle for the end of a pipette to rest. No part of the pipette needs to touch the delicate fins in the basin bottom or be hovered over them. Instead, the pipette end can be lodged against the corner, saving the bottom from potential puncture damage.

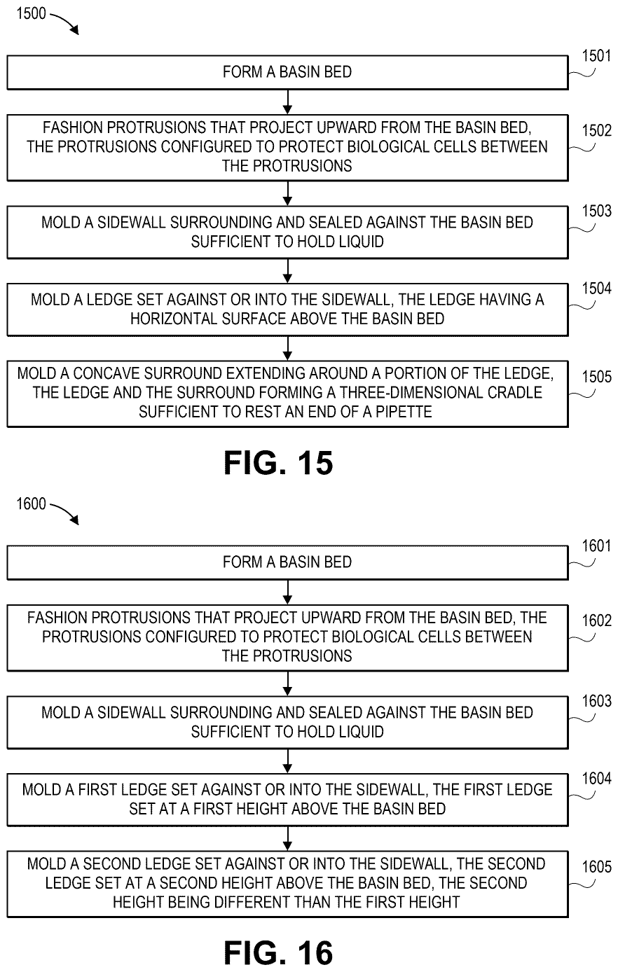

FIG. 15 is a flowchart illustrating process 1500 in accordance with an embodiment. In operation 1501, a basin bed is formed in a material. In operation 1502, protrusions are fashioned that project upward from the basin bed, the protrusions being configured to protect biological cells that can be grown between the protrusions. In operation 1503, a sidewall is molded that surrounds and seals against the basin bed sufficient to hold liquid. In operation 1504, a ledge that is set against or into the sidewall is molded, the ledge having a horizontal surface above the basin bed. In operation 1505, a concave surround is molded that extends around a portion of the ledge, the ledge and the surround forming a three-dimensional cradle sufficient to rest an end of a pipette. The fashioning and molding operations can be performed all at one time or at separate times and in various orders.

FIG. 16 is a flowchart illustrating process 1600 in accordance with an embodiment. In operation 1601, a basin bed is formed in a material. In operation 1602, protrusions are fashioned that project upward from the basin bed, the protrusions configured to protect biological cells that can be grown between the protrusions. In operation 1603, a sidewall is molded that surrounds and seals against the basin bed sufficient to hold liquid. In operation 1604, a first ledge set against or into the sidewall is molded, the first ledge set at a first height above the basin bed. In operation 1605, a second ledge set against or into the sidewall is molded, the second ledge set at a second height above the basin bed, the second height being different than the first height. The fashioning and molding operations can be performed all at one time or at separate times and in various orders.

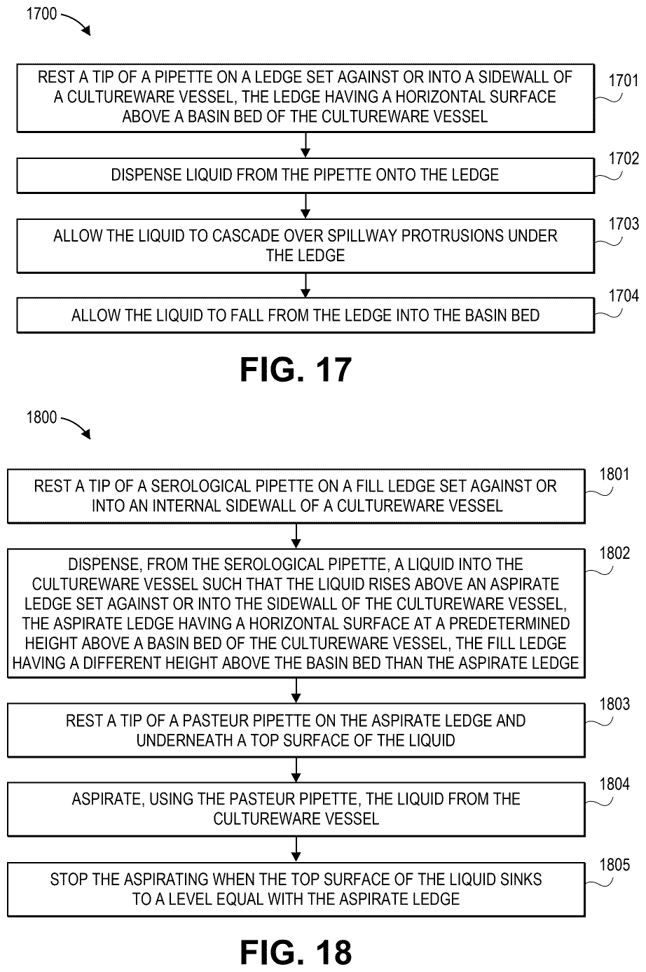

FIG. 17 is a flowchart illustrating process 1700 in accordance with an embodiment. In operation 1701, a tip of a pipette is rested on a ledge set against or into a sidewall of a cultureware vessel, the ledge having a horizontal surface above a basin bed of the cultureware vessel. In operation 1702, liquid is dispensed from the pipette onto the ledge. In operation 1703, the liquid is allowed to cascade over spillway protrusions under the ledge. In operation 1704, the liquid is allowed to fall from the ledge and protrusions into the basin bed.

FIG. 18 is a flowchart illustrating process 1800 in accordance with an embodiment. In operation 1801, a tip of a serological pipette is rested on a fill ledge set against or into an internal sidewall of a cultureware vessel. In operation 1802, a liquid is dispensed from the serological pipette into the cultureware vessel such that the liquid rises above an aspirate ledge set against or into the sidewall of the cultureware vessel, the aspirate ledge having a horizontal surface at a predetermined height above a basin bed of the cultureware vessel, the fill ledge having a different height above the basin bed than the aspirate ledge. In operation 1803, a tip of a Pasteur pipette is rested on the aspirate ledge and underneath a top surface of the liquid. In operation 1804, the liquid is aspirated from the cultureware vessel using the Pasteur pipette. In operation 1805, aspirating is stopped when the top surface of the liquid sinks to a level equal with the aspirate ledge.

Such a method can be used for cell seeding. Cells can be inoculated into the device utilizing any shelf that is below the media fill and mixed to evenly suspend cells within the device. Any remaining volume can be topped up with media. Media can be exchanged by aspirating it from the device and using the shelves as a reference or weir, and subsequently adding media to the device using the shelves as a reference.

Cells can reach the carrying capacity of the device at some point during expansion and may necessitate a culture passage. The lowest shelf can be utilized to aspirate the majority of the well volume without disturbing the growing cell population. The lowest shelf can also act to protect cells from the addition of liquids, such as phosphate-buffered saline (PBS), to wash the cells of residual media used during routine cell culture.

Some embodiments relate to the use of shelf-containing dishes, tray, plates, and devices for precise transfer of foreign deoxyribonucleic acid (DNA) to a cell, either through viral or non-viral means.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” in reference to a temperature or other engineering units includes measurements or settings that are within ±1%, ±2%, ±5%, ±10%, or other tolerances of the specified engineering units as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A cultureware vessel apparatus comprising:
a basin bed;
a sidewall surrounding and sealed against the basin bed sufficient to hold liquid;
a ledge set against or into the sidewall, the ledge having a horizontal surface above the basin bed; and
a concave surround extending around a portion of the ledge, the ledge and the surround forming a three-dimensional cradle sufficient to rest an end of a pipette.

2. The apparatus of claim 1 wherein the basin bed includes a resilient membrane comprised of a gas-permeable material and thin enough to allow oxygen permeation through the membrane.

3. The apparatus of claim 1 further comprising:
protrusions projecting upward from the basin bed, the protrusions configured to protect biological cells between the protrusions.

4. The apparatus of claim 3 wherein the protrusions include rows of parallel fins with grooves therebetween configured to protect biological cells.

5. The apparatus of claim 4 wherein the ledge is located perpendicular to the rows or at an end of the rows.

6. The apparatus of claim 1 wherein the ledge is a first ledge and the concave surround is a first concave surround, the apparatus further comprising:
a second ledge set against or into the sidewall, the second ledge having a horizontal surface above the basin bed; and
a second concave surround extending around a portion of the second ledge, the second ledge and the second surround forming a three-dimensional cradle sufficient to rest the end of the pipette.

7. (canceled)

8. The apparatus of claim 6 wherein the first and second ledges are located at different heights from each other.

9. The apparatus of claim 8 wherein the first ledge is directly above the second ledge.

10. (canceled)

11. The apparatus of claim 6 wherein the basin bed is circular, and the first and second ledges are set into the internal surface of the sidewall, the apparatus further comprising:
a third ledge set into the sidewall; and
a fourth ledge set into the sidewall,
wherein the ledges are located at 90° to one another around a circumference of the basin bed.

12. (canceled)

13. The apparatus of claim 6 wherein the basin bed is a polygon, and the first and second ledges are in corners of the polygon.

14. A T-flask comprising:
the apparatus of claim 13 wherein first and second ledges are set underneath one or more caps.

15. A multiwell culture plate comprising:
2, 3, 4, 6, 12, 24, 96, 384, or 1536 of the apparatuses of claim 1 integrally formed into a common molded polymer.

16. The multiwell culture plate of claim 15 wherein the molded polymer is an elastomer, the multiwell culture plate further comprising:
a rigid frame configured to mate with the elastomer and hold the basin beds of the apparatuses off of an underlying surface.

17. The apparatus of claim 1 wherein the surround is entirely inset into the sidewall.

18. The apparatus of claim 17 wherein a portion of the ledge protrudes from the sidewall to form a proscenium.

19. (canceled)

20. The apparatus of claim 1 wherein the horizontal surface of the ledge is sloped toward or away from the basin bed.

21. A method of manufacturing a cultureware vessel, the method comprising:
forming a basin bed;
molding a sidewall surrounding and sealed against the basin bed sufficient to hold liquid;
molding a ledge set against or into the sidewall, the ledge having a horizontal surface above the basin bed; and
molding a concave surround extending around a portion of the ledge, the ledge and the surround forming a three-dimensional cradle sufficient to rest an end of a pipette.

22-23. (canceled)

24. A cultureware vessel apparatus comprising:
a basin bed;
a sidewall surrounding and sealed against the basin bed sufficient to hold liquid;
a first ledge set against or into the sidewall, the first ledge set at a first height above the basin bed; and
a second ledge set against or into the sidewall, the second ledge set at a second height above the basin bed, the second height being different than the first height.

25-27. (canceled)

28. The apparatus of claim 27 wherein the first ledge is located perpendicular to the rows or at an end of the rows.

29. The apparatus of claim 24 wherein the first and second ledges share a common wall.

30-45. (canceled)
